(19) United States
(12) Patent Application Publication
(10) Pub. No.: US 2023/0070226 A1
(43) Pub. Date: Mar. 9, 2023

(54) MACROMOLECULES ENGINEERED FOR NANOELECTRONIC MEASUREMENT

(71) Applicant: Universal Sequencing Technology Corporation, Canton, MA (US)

(72) Inventors: Sanjay B. Hari, Sharon, MA (US); Peiming Zhang, Gilbert, AZ (US); Barrett Duan, Reading, MA (US); Ming Lei, Sharon, MA (US)

(73) Assignee: Universal Sequencing Technology Corporation, Canton, MA (US)

(21) Appl. No.: 17/759,742

(22) PCT Filed: Jan. 31, 2021
(86) PCT No.: PCT/US2021/015965

§ 371 Date: Jul. 29, 2022

Related U.S. Application Data
(60) Provisional application No. 62/968,929, filed on Jan. 31, 2020.

(51) Int. Cl. C12Q 1/6825; C12Q 1/6869; G01N 33/487; G01N 33/543; G16B 30/10; C12Q 1/6876

(52) U.S. Cl. CPC C12Q 1/6825; C12Q 1/6869; G01N 33/48721; G01N 33/5438; G16B 30/10; C12Q 1/6876

(57) ABSTRACT

The present invention provides methods to engineer enzymes for their integration into a molecular nanowire as a fum-tional component for biopolymer sequencing/identification. The enzymes include but are not limited to DNA polymerase, RNA poly-merase, DNA helicase, DNA ligase, DNA exonuclease, reverse transcriptase, RNA primase, ribosome, sucrase, or lactase, which are either natural, mutated, or synthesized.

### CROSS REFERENCE

This application claims the benefit of U.S. Provisional Patent Application No. 62/968,929, filed Jan. 31, 2020, whichi is incorporated by reference herein in its entirety.

### FIELD

This disclosure relates to biomolecules engineered for integrating into electronic circuits for biopolymer sensing/identification or sequencing.

### BACKGROUND

Polymeric macromolecules commonly found in biological systems generally comprise a defined set of building blocks linked in a specific order, so-called sequence. The sequence defines a polymer's three-dimensional structure and its functions in a biological system. In the case of proteins, this function can be an enzymatic reaction or binding event; in the case of carbohydrates, this function can be a recognition element. In the case of nucleic acids, this function can be a carrier of heritable information. Therefore, an accurate determination of the sequence of a polymeric macromolecule is critical to understanding its functions.

In the specific case of nucleic acids, the first generation of deoxyribonucleic acid (DNA) sequencing technology (“Sanger sequencing”) employed a method analyzing polymerization products from enzymatic reactions performed in bulk solution [1]. Read lengths for this technology under ideal conditions can reach 1000 base pairs (bp) or more. This approach was used for the international Human Genome Project, taking over ten years and costing ˜2.7 billion US dollars to generate the first human genome sequence [2, 3]. This technology is unfeasible as a tool for a large scale of genomics, albeit suitable for sequencing small genetic elements such as circular plasmids. The next generation sequencing (NGS) technologies were developed towards a $1000 genome and have reduced the cost and time to sequence a human genome [4, 5]. However, NGS is hindered by complicated structure variations and repeat sequences in the human genome due to its short read length.

Furthermore, because NGS is less accurate than Sanger sequencing, it more often requires deep sequencing, especially for determining mutations. An NGS variation that uses labeled enzymes instead of labeled nucleotides still only produces short reads [6].

To this end, the third generation sequencing technologies have been developed, which decode nucleic acids at the single-molecule level. For example, Pacific Biosciences sequencing platforms use zero-mode waveguides (ZMW), which detect fluorescence signals emitted by individual incorporation events [7]. This technology can read long DNA sequences, but it suffers from relatively high error rates. Thus, a sequencing platform with greater accuracy, more straightforward analysis, and lower deployment costs is desired in a wide range of applications, including personalized medicine and epidemiology.

Other approaches employ nanopores for sequencing. Biological nanopores, such as those marketed by Oxford Nanopore Technologies, use transmembrane protein pores [8]. Although this technology offers increased read lengths, it also suffers from low accuracy and is therefore often used in conjunction with NGS. Biological nanopore chips are costly to manufacture, hindering affordable sequencing and widespread deployment.

Solid-state nanopores in inorganic materials created by semiconductor technologies can be produced massively in a cost-effective fashion [9]. However, the geometry of solid-state nanopores cannot be controlled as precisely as that of biological pores. Therefore, a sensing mechanism must be incorporated into the solid-state nanopore for sequencing instead of the measurement of ionic current.

Various arrangements of nanopores and biosensors have been described. One approach [10] is to use the biosensor to feed hybridization probes from nucleotide-triphosphate analogs into the nanopore to elicit a detectable response. Another method, described generally in [11], is to connect the two sides of the nanogap with a bridge molecule, which transmits conformational changes of the associated biosensor arising from nucleotide incorporation events. An ideal configuration of these components maximizes sensitivity and reproducibility.

The individual components of the system may also vary in composition. For example, bridges can comprise carbon nanotubes or a DNA nanowire, but the latter carries the distinct advantages of being chemically defined and functionalizable at discrete locations. However, the conductivity of a single DNA molecule is controversial, especially when its length exceeds 30 nm.

DNA polymerases from E. coli and bacteriophage phi29 (phi29 pol) are routinely employed as biosensors. Disclosures such as those found in [11], [12], [13], and [14] broadly cover myriad configurations of probes, biosensors, and linkers without detailed teaching on how to achieve them. For example, one suggested embodiment in [13] describes selective conjugation of a biosensor to probe using well-established thiol-maleimide coupling chemistry and acknowledges further that doing so would likely necessitate the removal of all other cysteine residues in the biosensor, which is not a trivial task. In the specific case of phi29pol, seven native cysteine residues would have to be mutated to other naturally occurred residues. Doing so presents a real challenge requiring considerable amounts of experiments, as enzymes are only marginally stable, and even a single point mutation can cause deleterious functional consequences [15, 16]. In other cases, cysteine residues are essential for enzyme structure or function. For example, papain protease employs a cysteine residue in its catalytic cycle [17]. As another example, antibodies commonly use disulfide bonds formed by cysteine residues to maintain their structures [18].

Disclosure [13] also describes an embodiment that employs genetically-incorporated unnatural amino acids to facilitate conjugation, citing “click chemistry” as a non-limiting example. Several different types of bio-compatible conjugation chemistries have been described; however, to determine which one(s) is best suited to link a biosensor to a probe requires a significant amount of experiments.

Another example of problematic disclosure is found in [12], whereby the biosensor is attached using multiple linkage points to facilitate enhanced sensitivity via close coupling of the biosensor to the probe. However, protein surfaces provide many potential sites for conjugation, and extensive experimentation is required to determine the optimum point or points of contact to the probe. Without a predetermined attachment point, one is not able to control the orientation of the enzymatic probe, which may have a remarkable effect on the probe function. Furthermore, increasing the number of attachment sites increases the configuration possibilities combinatorically.

Protein fusion tags have been used extensively in the prior art to enhance protein expression, solubility, and activity [19]. In the specific case of polymerases, fusing the protein Sso7d from Sulfolobus solfataricus has been shown to enhance the processivity of thermostable polymerases by maintaining the association with DNA [20]. In another example, glutathione-S-transferase (GST) has been fused to phi29pol to aid in purification, but doing so required the addition of trehalose to retain protein solubility [21]. Embodiments of polymerases that enhance expression and solubility without the use of such additives are desirable.

### BRIEF DESCRIPTION OF DRAWINGS

FIG. 1 illustrates the principle of an engineered protein as a sensing component in a molecular device.

FIG. 2 shows a DNA duo conjugated with a protein through a click anchor.

FIG. 3 shows the structures of selenocysteine and derivatives thereof incorporated into proteins for conjugation and immobilization.

FIG. 4 shows phenylalanine-derived unnatural amino acids incorporated into proteins for conjugation and immobilization

FIG. 5 shows lysine-derived unnatural amino acids incorporated into proteins for conjugation and immobilization.

FIG. 6 shows the configurations of engineered DNA in this invention in a schematic form.

FIG. 7 shows a DNA polymerase bearing a solubility domain with some native residues replaced by unnatural amino acids (displayed as space-filling models)

FIG. 8 shows the seven natural cysteine residues of the phi29 DNA polymerase (displayed as space-filling models)

FIG. 9 illustrates the structure of the DNA wire comprising a modified nucleotide.

FIG. 10 shows functional molecules used to modify DNA internally.

FIG. 11 shows the synthetic route for compounds 1007 and 1008.

FIG. 12 shows gel analysis images of two cysteine mutants of phi29 polymerase. A) SUMO-phi29 mutants C11A and C11V were eluted from a Ni-NTA column (EL1 and EL2) and analyzed by SDS-PAGE. Based on comparison to the molecular weight ladder (M) the upper band is a full-length product, and the lower band is a truncated product. B) Different amounts of SUMO-phi29 polymerase mutant C11V were assayed for activity as described in Methods and analyzed by agarose gel electrophoresis. WT is a wild-type SUMO-phi29 polymerase.

FIG. 13 shows the properties of SUMO-phi29 polymerase mutants containing a specific unnatural amino acid residue. A) Different amounts of SUMO-phi29 polymerase wild-type (WT) and Y369pAzF mutant were analyzed by SDS-PAGE. B) Different quantities of SUMO-phi29 polymerase mutant Y369pAzF were assayed for activity as described in Methods and analyzed by agarose gel electrophoresis. WT is wild-type SUMO-phi29 polymerase, and T is phi29 polymerase from Thermo Scientific. U indicates units. C) SUMO-phi29 polymerase mutants E33pAzF and Y369pAzF were incubated with different concentrations of PEG5K-DBCO molecule (numbers underneath mutants, μM) at 20° C. and analyzed by SDS-PAGE. D) phi29 polymerase mutant E33pAzF was incubated with indicated DBCO conjugates at 4° C. and analyzed by SDS-PAGE. “DNA” is a single-stranded DNA molecule pre-conjugated to a DBCO-PEG5-TFP ester via an internal amine

### SUMMARY OF THE INVENTION

The present invention provides methods to engineer enzymes for their integration into a molecular nanowire as a functional component for biopolymer sequencing/identification. The said enzymes include but are not limited to DNA polymerase, RNA polymerase, DNA helicase, DNA ligase, DNA exonuclease, reverse transcriptase, RNA primase, ribosome, sucrase, or lactase, which are either natural, mutated, or synthesized.

The biopolymer includes but is not limited to DNA, RNA, oligonucleotides, protein, peptides, polysaccharides, etc., which are either natural or synthesized; and the molecular nanowire includes, but are not limited to a double-strand DNA (dsDNA or DNA duplex), a DNA duo (two dsDNA), a DNA nanostructure as disclosed in [24], or a combination thereof. The DNA duo is a simple DNA nanostructure and has an increased conductivity compared to a single DNA duplex. Below, we use the DNA duo and DNA polymerase to illustrate the method of engineering an enzyme. The same approach or principle applies to a single DNA duplex and a DNA nanostructure, sequencing and/or identifying different biopolymers using enzymes as sensors.

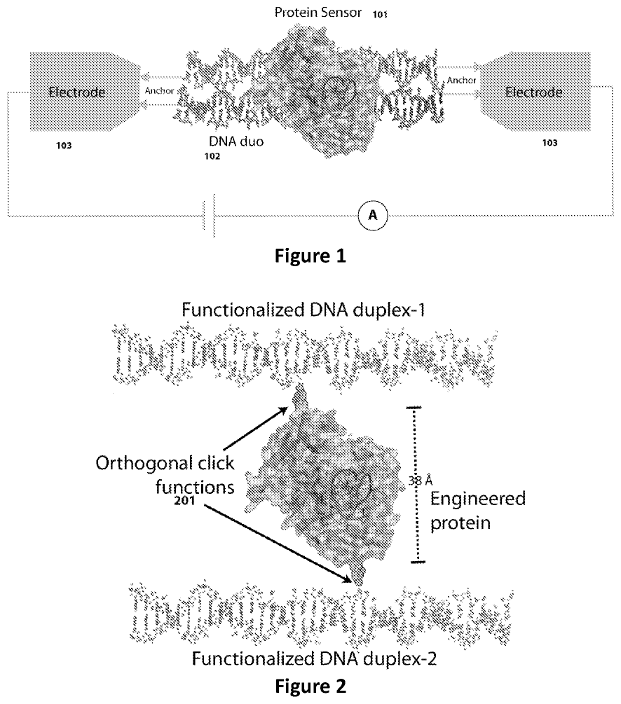

FIG. 1 shows a typical DNA sequencing device, in which a DNA polymerase (Protein Sensor 101) is attached to a DNA duo comprising a pair of double-stranded DNA molecules, referred to as DNA duo (102), are parallelly connected to two electrodes (103) that form a nanogap, where the gap size is from 2 nm to 1000 nm, preferably from 5 nm to 100 nm, and most preferably from 5 nm to 30 nm. This device can monitor the process of an enzyme catalyzing the incorporation of individual nucleoside triphosphates into a DNA primer along with a template in real-time by recording changes in the conductivity of the DNA nanowire caused by those chemical events. Thus, one application for such a device is the sequencing of nucleic acids at a single-molecule level. To enhance the sequencing throughput, these nanogap/nanowire devices can form an array in the size of about 100 to about 100 million, preferably 10,000 to 1 million, such as that described in [24]. It would provide benefits of, for example, longer read length, enhanced accuracy, and reduced cost of operation.

### DETAILED DESCRIPTION OF THE INVENTION

In one embodiment of the present invention, the said enzyme is an engineered DNA polymerase that carries unnatural amino acid residues containing an orthogonal functional group at two predefined positions (201, FIG. 2). It is explicitly attached to the DNA duo at predefined locations in the DNA duo so that the electrical currents fluctuate in concert with enzymatic activities. Two attachment points provide better control for the orientation of the polymerase to the DNA duo.

In some embodiments of the present invention, the said enzyme is a wild-type DNA polymerase engineered with unnatural amino acids at the pre-select sites (702, FIG. 7). The chosen locations provide the device with high sensitivity to sense the enzymatic events without interrupting the enzyme's catalytic activity. One example is placing one unnatural amino acid in the exonuclease domain and the other in the finger domain. Other examples include but are not limited to placing one unnatural amino acid in the finger domain, and the other location is chosen from the non-exclusive list of the palm, TPR1, thumb, and ΔTPR2 domains.

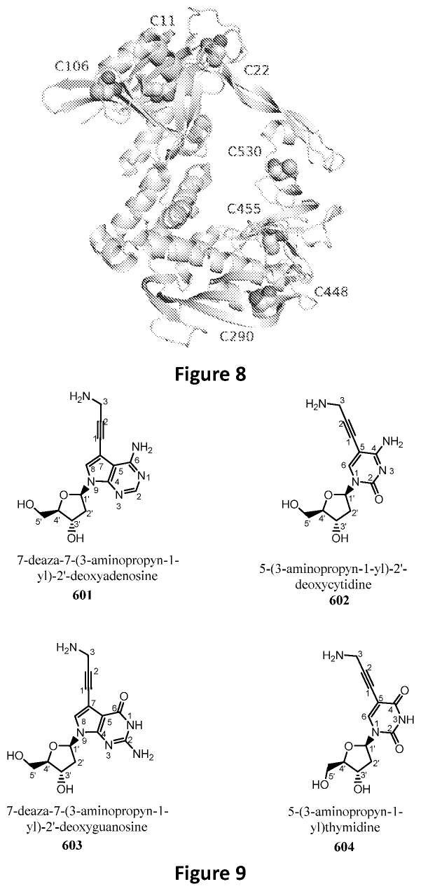

In some embodiments, the mutant DNA polymerase includes a fused, genetically-encoded protein conveying enhanced solubility and activity (701) (Sequence ID #1). The fused polymerase is engineered to contain only one or two cysteine residues (FIG. 8, Sequence ID #2), which allows the protein to react with thiol acceptors for bioconjugation in a site-specific manner Such mutants retain catalytic activity to function as a biosensor.

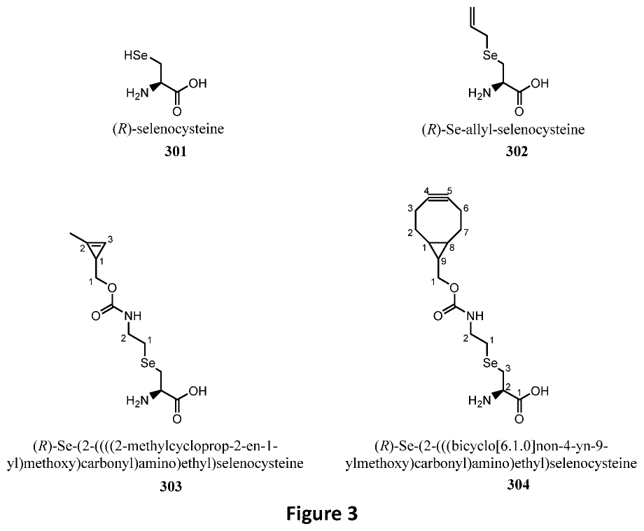

In some embodiments, the fused polymerase is engineered by replacing some of its cysteines with selenocysteine (301, FIG. 3) for selectively reacting with electrophiles at the desired site under slightly acidic conditions.

In some embodiments, the unnatural amino acid used for protein engineering is a derivative of selenocysteine (shown in FIG. 3, but not limited to them), which is incorporated into the said protein and mutants according to the cloning method stated in Methodology.

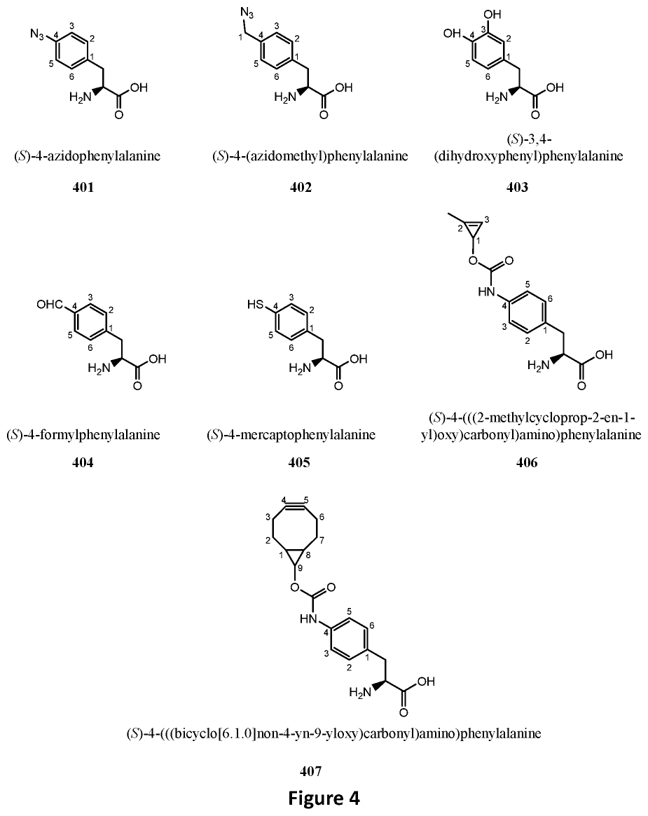

In some embodiments, the said unnatural amino acid is a derivative of natural phenylalanine, which is incorporated into the said protein and mutants according to the cloning method stated in Methodology. Some of the phenylalanine derivatives are shown in FIG. 4, but not limited to them.

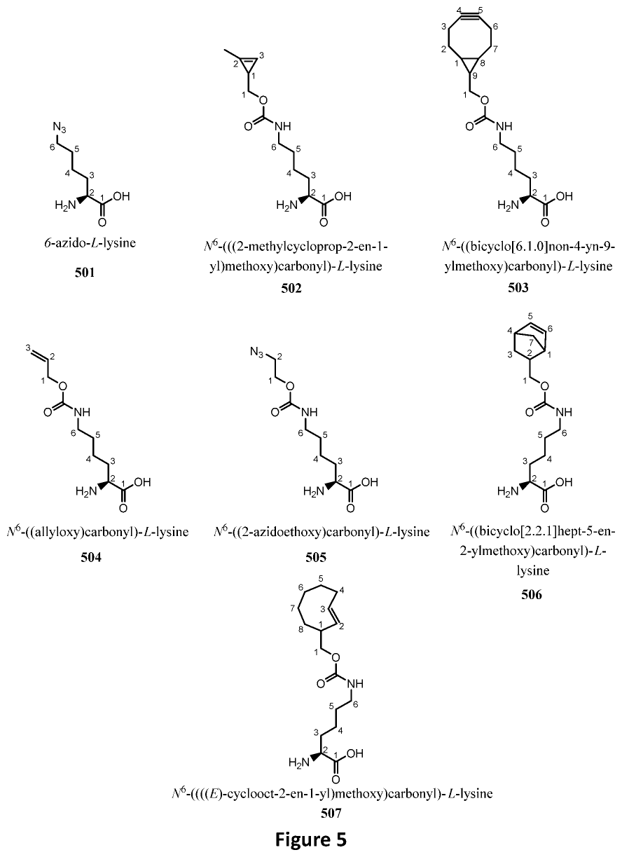

In some embodiments, the said unnatural amino acid is a derivative of natural lysine, which is incorporated into the said protein and mutants according to the cloning method stated in Methodology, Some of the lysine derivatives are shown in FIG. 5, but not limited to therein,

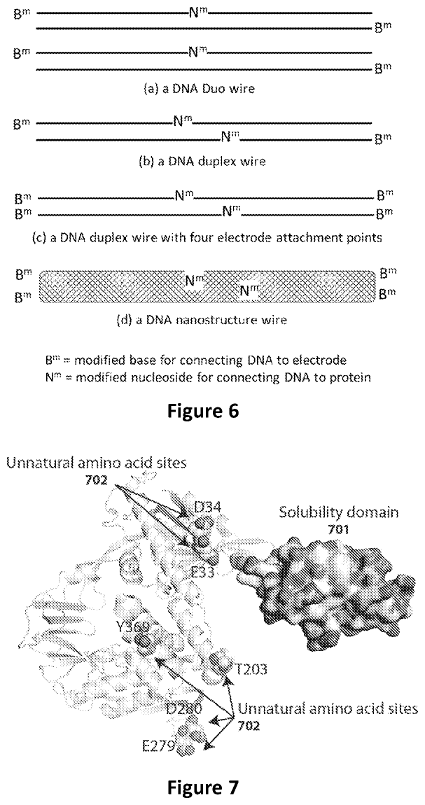

In some embodiments, this invention provides a DNA duo to form a molecular junction as a medium for incorporating the said protein or a mutant and conveying the protein's movement to electrical signals. Each DNA duplex has one nucleoside functionalized (N^{m}), able to react with one of the said unnatural amino acids in the engineered protein or polymerase in the case of DNA/RNA sequencing, and two functional groups (B^{m}) at its two ends for attaching to the two electrodes at the nanogap respectively (FIG. 6(a)).

In some embodiments, the said DNA junction is a single DNA duplex (dsDNA), each strand of which has one nucleoside functionalized (N^{m}), able to react with the said noncanonical and unnatural amino acids engineered into the said protein or polymerase in the case of DNA/RNA sequencing, and one or two functional groups (B^{m}) at each end of the duplex for attaching to the two electrodes at the nanogap (FIG. 6 (b) & (c)). In addition, the DNA sequence can be palindromic, allowing a duplex to form spontaneously in solution from an oligonucleotide.

In some embodiments, the said DNA junction is a DNA nanostructure as disclosed in [24, 25] and two predefined locations in the nanostructure have nucleosides functionalized (N^{m}), able to react with the said noncanonical and unnatural amino acids engineered into the said protein or polymerase in the case of DNA/RNA sequencing, and one or two functional groups (B^{m}) at each end of the DNA nanostructure for attaching to the two electrodes at the nanogap (FIG. 6 (d)).

In some embodiments, the double-stranded DNA has an amino function at one of its internal bases. For example, an amino group is situated at the 5-position of a pyrimidine base or the 7-position of a purine base. Some of these nucleosides are shown in FIG. 9, but not limited to them. These nucleosides can be converted to their respective phosphoramidites and be incorporated into DNA by an automated DNA synthesizer.

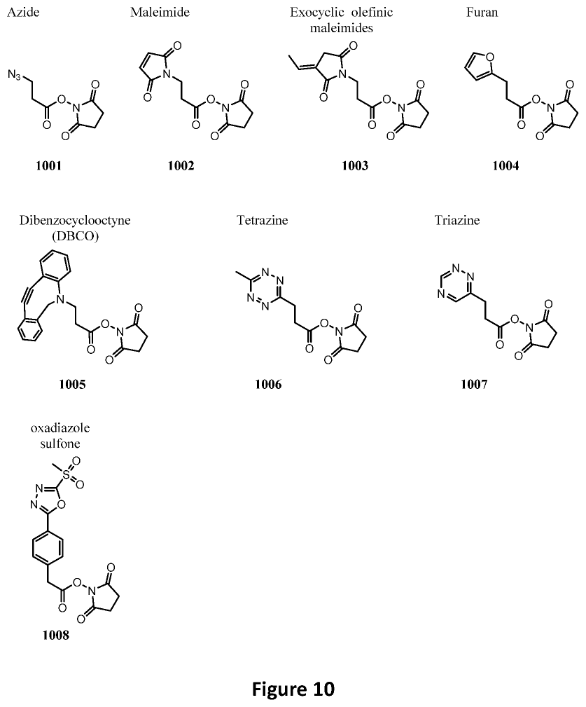

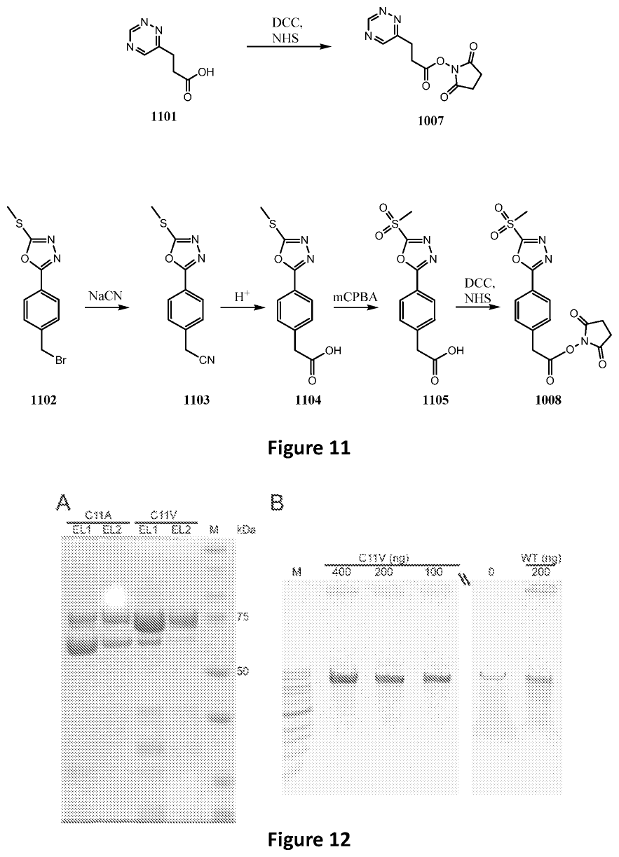

The aminated DNA is further functionalized with functional groups that can specifically react with the said unnatural amino acids engineered into the said protein or polymerase in the case of DNA/RNA sequencing. Some of which are shown in FIG. 10. Each of these compounds contains an N-hydroxysticcinimide (NHS) ester that can rapidly react with the alkylamine These compounds are commercially available except 1007 and 1008. Compounds 1007 and 1008 are synthesized by the methods shown in FIG. 11. First, 1007 is synthesized by 1,2,4-Triazine-6-propanoic acid (1101) reacting with N-hydroxysuccinimide (NHS) in the presence of dicyclohexylcarbodiimide (DCC). The compound 1008 is synthesized, stating from 2-(4-(bromomethyl)phenyl)-5-(methylthio)-1,3,4-oxadiazole (1102) [22] via four steps.

In some embodiments of the present invention, the DNA duo generally comprises two double-stranded DNA with a length that can bridge two electrodes separated by a distance ranging from 3 to 50 nanometer. In some other embodiments, the DNA duo is replaced by two double-stranded RNA, PNA, XNA, or hybrids of DNA to RNA, DNA to PNA, DNA to XNA, RNA to PNA, RNA to XNA, or PNA to XNA.

In some embodiments, the sequence of a DNA duplex, either alone or being part of a DNA duo or a DNA nanostructure, contains at least 50% of GC base pairs with a length ranging from 10 to 150 base pairs. Besides the canonical bases, the DNA duplex also includes modified nucleobases and/or base analogs for improving its conductivity.

In some embodiments, the DNA duo comprises the palindromic double-stranded DNA that is formed spontaneously in solution from a single-stranded oligonucleotide with a self-complementary sequence. Both double-stranded DNA molecules in the DNA duo have the same symmetry without polarity along their helical axes. When the DNA duo is used as a molecular wire to bridge the nanogap, its two ends can be attached to either one of two electrodes, which would not cause electrical polarities.

### Methodology

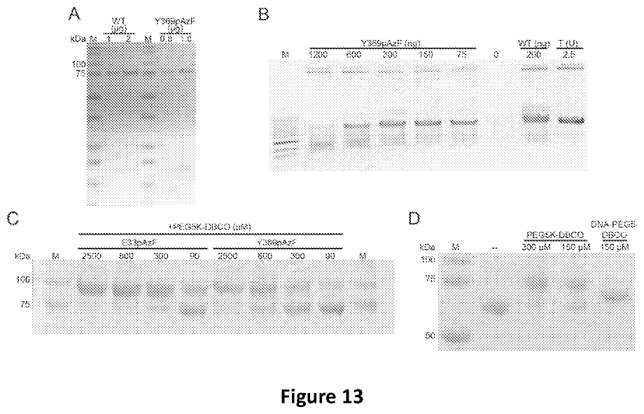

Cloning. A gene cassette harboring sequences encoding a fusion protein and wild-type DNA polymerase from phi29 (phi29pol) was inserted into a T7-based plasmid such as pET21a and expressed in E. coli. Point mutations were made using PCR with oligonucleotide primers containing desired mutations [23]. The recombinant protein was purified using Ni-NTA agarose. Typical yields are approximately 30 mg per liter of culture (FIG. 12A and 13A).

Activity assay. In a typical, non-limiting reaction, enzyme (100 ng) is incubated in a buffered solution containing plasmid DNA (20 ng), dNTPs, and single-stranded DNA primer at 30° C. Products are digested with EcoRI, separated by agarose gel electrophoresis, and visualized by fluorescence (FIG. 12B and 13B).

DNA-functionalization with DBCO. In a typical, non-limiting reaction, single-stranded DNA containing an amino function (50 μM) is incubated with DBCO-PEG5-TFP ester (2.5 mM) in sodium tetraborate buffer (pH 9) overnight at 25° C. Any unreacted linker is removed by ethanol precipitation.

Macromolecule-enzyme conjugation. In a typical, non-limiting reaction, enzyme (30 μM) containing a p-azidophenylalanine residue is incubated in a buffered solution containing DBCO-conjugated macromolecules (150 μM) molecule at 20° C. (FIG. 13C) or overnight at 4° C. (FIG. 13D).

Sequence listing

Sequence ID #1
Type: protein
Organism: synthetic sequence
Other information: DNA polymerase fusion protein
MGHHHHHHHDSEVNQEAKPEVKPEVKPETHINLKVSDGSSEIFFKIKKTTPLRRLMEAFAKRQGK
EMDSLRFLYDGIRIQADQTPEDLDMEDNDIIEAHREQIGGNGSKHMPRKMYSCDFETTTKVEDCR
VWAYGYMNIEDHSEYKIGNSLDEFMAWVLKVQADLYFHNLKFDGAFIINWLERNGFKWSADGLPN
TYNTIISRMGQWYMIDICLGYKGKRKIHTVIYDSLKKLPFPVKKIAKDFKLTVLKGDIDYHKERP
VGYKITPEEYAYIKNDIQIIAEALLIQFKQGLDRMTAGSDSLKGFKDIITTKKFKKVFPTLSLGL
DKEVRYAYRGGFTWLNDRFKEKEIGEGMVFDVNSLYPAQMYSRLLPYGEPIVFEGKYVWDEDYPL
HIQHIRCEFELKEGYIPTIQIKRSRFYKGNEYLKSSGGEIADLWLSNVDLELMKEHYDLYNVEYI
SGLKFKATTGLFKDFIDKWTYIKTTSEGAIKQLAKLMLNSLYGKFASNPDVTGKVPYLKENGALG
FRLGEEETKDPVYTPMGVFITAWARYTTITAAQACYDRIIYCDTDSIHLTGTEIPDVIKDIVDPK
KLGYWAHESTFKRAKYLRQKTYIQDIYMKEVDGKLVEGSPDDYTDIKFSVKCAGMTDKIKKEVTF
ENFKVGFSRKMKPKPVQVPGGVVLVDDTFTIKSA
Sequence ID #2
Type: protein
Organism: synthetic sequence
Other information: DNA polymerase fusion protein with a single
cysteine
MGHHHHHHHDSEVNQEAKPEVKPEVKPETHINLKVSDGSSEIFFKIKKTTPLRRLMEAFAKRQGK
EMDSLRFLYDGIRIQADQTPEDLDMEDNDIIEAHREQIGGNGSKHMPRKMYSADFETTTKVEDAR
VWAYGYMNIEDHSEYKIGNSLDEFMAWVLKVQADLYFHNLKFDGAFIINWLERNGFKWSADGLPN
TYNTIISRMGQWYMIDIALGYKGKRKIHTVIYDSLKKLPFPVKKIAKDFKLTVLKGDIDYHKERP
VGYKITPEEYAYIKNDIQIIAEALLIQFKQGLDRMTAGSDSLKGFKDIITTKKFKKVFPTLSLGL

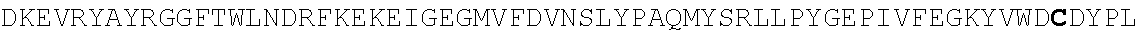

HIQHIRAEFELKEGYIPTIQIKRSRFYKGNEYLKSSGGEIADLWLSNVDLELMKEHYDLYNVEYI
SGLKFKATTGLFKDFIDKWTYIKTTSEGAIKQLAKLMLNSLYGKFASNPDVTGKVPYLKENGALG
FRLGEEETKDPVYTPMGVFITAWARYTTITAAQAAYDRIIYADTDSIHLTGTEIPDVIKDIVDPK
KLGYWAHESTFKRAKYLRQKTYIQDIYMKEVDGKLVEGSPDDYTDIKFSVKAAGMTDKIKKEVTF
ENFKVGFSRKMKPKPVQVPGGVVLVDDTFTIKSA
Sequence ID #3
Type: protein
Organism: synthetic sequence
Other information: DNA polymerase fusion protein with single
cysteine and single genetically-encoded non-canonical amino
acid
MGHHHHHHHDSEVNQEAKPEVKPEVKPETHINLKVSDGSSEIFFKIKKTTPLRRLMEAFAKRQGK
EMDSLRFLYDGIRIQADQTPEDLDMEDNDIIEAHREQIGGNGSKHMPRKMYSADFETTTKVEDAR
VWAYGYMNIEDHSEYKIGNSLDEFMAWVLKVQADLYFHNLKFDGAFIINWLERNGFKWSADGLPN
TYNTIISRMGQWYMIDIALGYKGKRKIHTVIYDSLKKLPFPVKKIAKDFKLTVLKGDIDYHKERP
VGYKITPEEYAYIKNDIQIIAEALLIQFKQGLDRMTAGSDSLKGFKDIITTKKFKKVFPTLSLGL

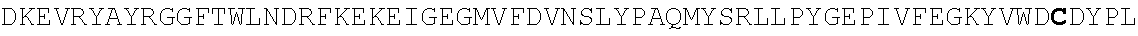

HIQHIRAEFELKEGYIPTIQIKRSRFYKGNEYLKSSGGEIADLWLSNVDLELMKEHYDLYNVEYI

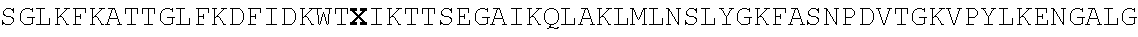

FRLGEEETKDPVYTPMGVFITAWARYTTITAAQAAYDRIIYADTDSIHLTGTEIPDVIKDIVDPK
KLGYWAHESTFKRAKYLRQKTYIQDIYMKEVDGKLVEGSPDDYTDIKFSVKAAGMTDKIKKEVTF
ENFKVGFSRKMKPKPVQVPGGVVLVDDTFTIKSA
Sequence ID #4
Type: protein
Organism: synthetic sequence
Other information: DNA polymerase fusion protein with two
genetically-encoded non-canonical amino acids
MGHHHHHHHDSEVNQEAKPEVKPEVKPETHINLKVSDGSSEIFFKIKKTTPLRRLMEAFAKRQGK
EMDSLRFLYDGIRIQADQTPEDLDMEDNDIIEAHREQIGGNGSKHMPRKMYSADFETTTKVEDAR
VWAYGYMNIEDHSEYKIGNSLDEFMAWVLKVQADLYFHNLKFDGAFIINWLERNGFKWSADGLPN
TYNTIISRMGQWYMIDIALGYKGKRKIHTVIYDSLKKLPFPVKKIAKDFKLTVLKGDIDYHKERP
VGYKITPEEYAYIKNDIQIIAEALLIQFKQGLDRMTAGSDSLKGFKDIITTKKFKKVFPTLSLGL

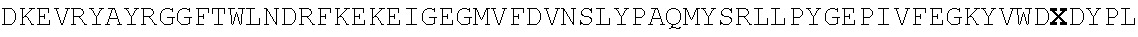

HIQHIRAEFELKEGYIPTIQIKRSRFYKGNEYLKSSGGEIADLWLSNVDLELMKEHYDLYNVEYI

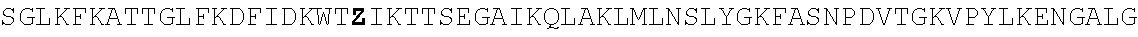

FRLGEEETKDPVYTPMGVFITAWARYTTITAAQAAYDRIIYADTDSIHLTGTEIPDVIKDIVDPK
KLGYWAHESTFKRAKYLRQKTYIQDIYMKEVDGKLVEGSPDDYTDIKFSVKAAGMTDKIKKEVTF
ENFKVGFSRKMKPKPVQVPGGVVLVDDTFTIKSA

Claimable items of this invention include, but not limited to, the following:

An embodiment is a DNA duplex or a DNA duo that bridges a nanogap between two electrodes. The said DNA duplex or DNA duo comprises:
a. Double-stranded DNA molecules, either in A-form, or B-form or Z-form.
b. Double-stranded nucleic acid helices including those natural and non-natural.
c. Double-stranded molecules connected through a biomolecule.
d. Double-stranded molecules containing linkers at their ends.
e. Double-stranded molecules containing internal functional groups for attaching recognition molecules including those with a molecular weight ranging from 100 to 200,000 Da.
f. Double-stranded DNA containing modified nucleotides that increase the conductivity of the double-stranded DNA, as disclosed in [25], such as a single nucleic acid duplex (double strands), a nucleic acid triplex, a nucleic acid quadruplex, a nucleic acid origami structure, and a combination thereof, wherein the nucleic acid bases are either natural, modified or synthesized or the combination thereof.

An embodiment is a functional protein engineered to at least contain one of the above said noncanonical amino acid residues at predefined positions.
a. The said protein fused to another protein with enhanced solubility and stability.
b. The said protein spontaneously and precisely forming covalent connections with an engineered molecular wire.

An embodiment is a functional protein engineered to contain two of the above said noncanonical amino acid residues at the predefined positions, and the said protein spontaneously and precisely forming covalent connections at two predefined positions on an engineered molecular wire.

An embodiment is a method to label enzymes with biomolecules and organic molecules.

An embodiment is the DNA duplex or DNA duo or DNA nanostructure internally carrying a nucleophile capable of reacting with the above said NHS, PFP, or TFP esters of functional molecules or other chemically active species.
a. The said molecular wire has a length of ranging from 2 to 1000 nm, preferably 5 to 100 nm, most preferably 5 to 30 nm.
b. The said molecular wires spontaneously and precisely forming covalent connections with engineered proteins.

An embodiment is a method to engineer DNA with different functional groups at predetermined locations.

### General Remarks

All publications, patents, and other documents mentioned herein are incorporated by reference in their entirety.

Unless defined otherwise, all technical and scientific terms used herein have the same meaning as those commonly understood by one of ordinary skill in the art to which this invention belongs. While the present invention has been illustrated by a description of various embodiments and while these embodiments have been described in considerable detail, it is not the intention of the applicants to restrict or in any way limit the scope of the applications. Additional advantages and modifications will readily appear to those skilled in the art. The invention in its broader aspects is therefore not limited to the specific details, representative device, apparatus and method, and illustrative example shown and described. Accordingly, departures may be made from such details without departing from the spirit of the applicant's general inventive concept.

### References

1. Smith L M, Sanders J Z, Kaiser R J, Hughes P, Dodd C, Connell C R, et al. Fluorescence detection in automated DNA sequence analysis. Nature. 1986; 321: 674-9.

2. Lander E S, Linton L M, Birren B, Nusbaum C, Zody M C, Baldwin J, et al. Initial sequencing and analysis of the human genome. Nature. 2001; 409: 860-921.

3. Venter J C, Adams M D, Myers E W, Li P W, Mural R J, Sutton G G, et al. The sequence of the human genome. Science. 2001; 291: 1304-51.

4. Margulies M, Egholm M, Altman W E, Attiya S, Bader J S, Bemben L A, et al. Genome sequencing in microfabricated high-density picolitre reactors. Nature. 2005; 437: 376-80.

5. Turcatti G, Romieu A, Fedurco M, Tairi A P. A new class of cleavable fluorescent nucleotides: synthesis and optimization as reversible terminators for DNA sequencing by synthesis. Nucleic Acids Res. 2008;36: e25.

6. Previte M J, Zhou C, Kellinger M, Pantoja R, Chen C Y, Shi J, et al. DNA sequencing using polymerase substrate-binding kinetics. Nat Commun. 2015; 6: 5936.

7. Eid J, Fehr A, Gray J, Luong K, Lyle J, Otto G, et al. Real-time DNA sequencing from single polymerase molecules. Science. 2009; 323: 133-8.

8. Stoddart D, Heron A J, Mikhailova E, Maglia G, Bayley H. Single-nucleotide discrimination in immobilized DNA oligonucleotides with a biological nanopore. Proc Natl Acad Sci U S A. 2009; 106: 7702-7.

9. Dekker C. Solid-state nanopores. Nat Nanotechnol. 2007; 2: 209-15.

10. Mandell J G, Gunderson, Kevin L., Gundlach, Jens H. Compositions, systems, and methods for detecting events using tethers anchored to or adjacent to nanopores. The United States Patent Application No. 20190376135, 2019.

11. Merriman BLSD, Mola, Paul W., Biomolecular sensors and methods. The United States Patent Application No. 20180340220, 2018.

12. Merriman B L, Govindaraj V A, Mola P., Geiser T. ENZYMATIC CIRCUITS FOR MOLECULAR SENSORS. The United States Patent Application No. 20180305727, 2018.

13. Merriman B L, Govindaraj V A, Mola P., Geiser T., Costa G. BINDING PROBE CIRCUITS FOR MOLECULAR SENSORS. The United States Patent Application No. 20190004003, 2019.

14. Merriman B L S D, Mola P., Choi C. MOLECULAR SENSORS AND RELATED METHODS. The United States Patent Application No. 20190094175, 2019.

15. Matthews B W. Studies on protein stability with T4 lysozyme. Adv Protein Chem. 1995; 46: 249-78.

16. Yutani K, Ogasahara K, Tsujita T, Sugino Y. Dependence of conformational stability on hydrophobicity of the amino acid residue in a series of variant proteins substituted at a unique position of tryptophan synthase alpha subunit. Proc Natl Acad Sci USA. 1987; 84: 4441-4.

17. Klein I B, Kirsch J F. The activation of papain and the inhibition of the active enzyme by carbonyl reagents. J. Biol. Chem. 1969; 244: 5928-35.

18. Liu H, May K. Disulfide bond structures of IgG molecules: structural variations, chemical modifications and possible impacts to stability and biological function. MAbs. 2012; 4: 17-23.

19. Costa S, Almeida A, Castro A, Domingues L. Fusion tags for protein solubility, purification and immunogenicity in Escherichia coli: the novel Fh8 system. Front Microbiol. 2014; 5: 63.

20. Wang Y, Prosen D E, Mei L, Sullivan J C, Finney M, Vander Horn P B. A novel strategy to engineer DNA polymerases for enhanced processivity and improved performance in vitro. Nucleic Acids Res. 2004; 32: 1197-207.

21. Takahashi H, Yamazaki H, Akanuma S, Kanahara H, Saito T, Chimuro T, et al. Preparation of Phi29 DNA polymerase free of amplifiable DNA using ethidium monoazide, an ultraviolet-free light-emitting diode lamp and trehalose. PLoS One. 2014; 9: e82624.

22. Chen B, Long Q, Zhao Y, Wu Y, Ge S, Wang P, et al. Sulfone-Based Probes Unraveled Dihydrolipoamide S-Succinyltransferase as an Unprecedented Target in Phytopathogens. Journal of Agricultural and Food Chemistry. 2019; 67: 6962-9.

23. Liu H, Naismith J H. An efficient one-step site-directed deletion, insertion, single and multiple-site plasmid mutagenesis protocol. BMC Biotechnol. 2008; 8: 91.

24. Zhang P, Lei M, Devices, methods and chemical reagents for biopolymer sequencing. The U.S. Patent Application No. 62/794,096, 2019.

25. Zhang P, Krstic P, Lei M, Engineered DNA for molecular electronics, U.S. Patent Application No. 62/938,084, 2019

1. A system for identification, characterization, or sequencing of a biopolymer comprising,
a. a nanogap formed by a first electrode and a second electrode placed next to each other;
b. a nucleic acid molecular wire with a length comparable to the nanogap that bridges the nanogap by attaching one end of the molecular wire to the first electrode and another end of the molecular wire to the second electrode each through a chemical bond, wherein two internal nucleosides within the molecular wire at pre-defined positions are functionalized, allowing the attachment of a protein or a sensing molecule, and wherein the molecular wire has one or more attachment sites at each end; and
c. a sensing probe with two attachment sites attached to the two corresponding functionalized sites on the molecular wire that can interact or perform a chemical or a biochemical reaction with the biopolymer, wherein the two attachment sites interact with the two functionalized sites on the molecular wire and control the orientation of the sensing probe.

2. The system of claim 1, further comprising,
a. a bias voltage that is applied between the first electrode and the second electrode;
b. a device that records a current fluctuation through the molecular wire caused by the interaction between the sensing probe and the biopolymer; and
c. a software for data analysis that identifies or characterizes the biopolymer or a subunit of the biopolymer.

3. The system of claim 1, wherein the biopolymer is selected from the group consisting of a DNA, an RNA, a protein, a carbohydrate, a polypeptide, an oligonucleotide, a polysaccharide, and their analogues, either natural, synthesized, modified, and a combination thereof.

4. The system of claim 1, wherein the sensing probe is selected from the group consisting of a nucleic acid probe, an enzyme, a receptor, a ligand, an antigen and an antibody, either native, mutated, synthesized, and a combination thereof.

5. The system of claim 4, wherein the enzyme is selected from the group consisting of a DNA polymerase, an RNA polymerase, a DNA helicase, a DNA ligase, a DNA exonuclease, a reverse transcriptase, an RNA primase, a ribosome, a sucrase, lactase, either natural, mutated, synthesized, and a combination thereof.

6. The system of claim 4, wherein the enzyme is engineered to comprise an unnatural amino acid at a pre-defined site.

7. The system of claim 6, wherein the unnatural amino acid used for protein engineering is a selenocysteine or a phenylalanine or a lysine or a derivative thereof, either natural, synthesized, mutated, or a combination thereof

8. The system of claim 5, wherein the two engineered sites on the DNA or RNA polymerase are configured with one site in a finger domain, and the other site in either an exonuclease, or a palm, or a thumb, or a TPR1 or a DTPR2 domain.

9. The system of claim 5, wherein the DNA or RNA polymerase is engineered to comprise only one or two cysteine residues for attachment to the molecular wire.

10. The system of claim 5, wherein the DNA or RNA polymerase is engineered to comprise at least a selenocysteine or wherein at least one cysteine therein is replaced with a selenocysteine.

11. The system of claim 1, wherein the molecular wire is selected from the group consisting of a single nucleic acid duplex, a nucleic acid duplex duo, a nucleic acid triplex, a nucleic acid quadruplex, a nucleic acid origami structure, and a combination thereof wherein the nucleic acid strand is either in an A-form, a B-form or a Z-form and the nucleic acid bases are either natural or unnatural.

12. The system of claim 11, wherein the single nucleic acid duplex comprises a functionalized nucleic acid base at a pre-defined position on each strand and one attachment site at the end of each duplex or at the end of each strand; and the nucleic acid duplex duo has one functionalized nucleic base on each duplex and one attachment site at the end of each duplex.

13. The system of claim 11, wherein the sequence of a nucleic acid duplex is palindromic.

14. The system of claim 1, wherein the nucleic acid molecular wire comprises an amino function at one of its internal bases at a pre-defined position,

15. The system of claim 14, wherein the base with amino function is further functionalized with a moiety carrying an activated carboxylate, including but not limited to an azide, a maleimide, an exocyclic olefinic maleimide, a furan, a dibenzocyclooctane, a tetrazine, a triazine, an oxadiazole sulfone.

16. The system of claim 11, wherein the nucleic acid duplex duo comprises two double-stranded PNA, XNA or a hybrid of DNA/RNA, DNA/PNA, DNA/XNA, RNA/PNA, RNA/XNA or PNA/XNA, either natural, modified, synthesized, or a combination thereof, or is replaced by two double-stranded PNA, XNA or a hybrid of DNA/RNA, DNA/PNA, DNA/XNA, RNA/PNA, RNA/XNA or PNA/XNA, either natural, modified, synthesized, or a combination thereof.

17. The system of claim 1, wherein the nucleic acid molecular wire comprises at least 50% of GC base pairs.

18. The system of claim 1, wherein the nanogap size or the distance between the ends of the two electrodes is about 2 to 1000 nm, or about 5 to 100 nm, or about 5 to 30 nm.

19. The system of claim 1, wherein the nanogap comprises a plurality of nanogaps, each comprising a pair of electrodes, a molecular wire, a sensing probe, and any feature associated with a single nanogap.

20. A method for identification, characterization, or sequencing of a biopolymer comprising,
a. forming a nanogap by placing a first electrode and a second electrode placed next to each other;
b. providing a nucleic acid molecular wire with a length comparable to the nanogap, wherein two internal nucleosides of the molecular wire at pre-defined positions are functionalized, allowing the attachment of a protein or a sensing molecule, and wherein the molecular wire has one or more attachment sites at each end;
c. providing a sensing probe that can interact or perform a chemical or a biochemical reaction with the biopolymer, wherein the sensing probe has two attachment sites that can interact with the two functionalized sites on the molecular wire;
d. attaching one end of the molecular wire to the first electrode and another end of the molecular wire to the second electrode through attachment sites at the end of the molecular wire; and
e. attaching the sensing probe to the molecular wire through the two attachment sites on the sensing probe and the two functionalized sites on the molecular wire.
wherein step “e” could occur before step “d” or vice versa.

21-38. (canceled)
